(19) United States
(12) Patent Application Publication
(10) Pub. No.: US 2007/0233062 A1
(43) Pub. Date: Oct. 4, 2007

(54) PEDICLE SCREW SYSTEM WITH OFFSET STABILIZER ROD

(75) Inventor: Bret M. Berry, Sandy, UT (US)

Correspondence Address: KELLY LOWRY & KELLEY, LLP, 6320 CANOGA AVENUE, SUITE 1650, WOODLAND HILLS, CA, 91367, US

(73) Assignee: AMEDICA CORPORATION, Salt Lake City, UT (US)

(21) Appl. No.: 11/308,544

(22) Filed: Apr. 4, 2006

(51) Int. Cl. A61F 2/44

(52) U.S. Cl. 606/61; 606/64

(57) ABSTRACT

An improved pedicle screw system is provided with an offset stabilizer rod for the internal fixation of the spine. The pedicle screw system includes at least two multi-angle pedicle screw units adapted for anchored securement to patient bone, and an elongated stabilizer rod extending therebetween. Each pedicle screw unit includes a bone screw associated with an anchor bracket defining a laterally offset and upwardly open channel or trough for receiving and supporting the stabilizer rod. A securement member such as a set screw is fastened to the anchor bracket for compressively retaining the stabilizer rod within the bracket channel or trough. The securement member may also bear against the associated bone screw for compressively retaining the screw in position relative to the anchor bracket.

### BACKGROUND OF THE INVENTION

This invention relates generally to improvements in devices and systems used for internal fixation of adjacent vertebral bodies of the spine. More specifically, this invention relates to an improved and relatively low profile pedicle screw system having a laterally offset stabilizer rod adapted for facilitated and highly stable implantation.

The spinal column is a highly complex system of bones and connective tissues that provides support for the body and protects the delicate spinal column and nerves. The spinal column includes a series of vertebrae stacked one atop the other, whereby each vertebral body includes a relatively strong bone portion (cortical) forming the outside surface of the body and a relatively weak bone portion (cancellous) forming the center of the body. Situated between each vertebral body is an intervertebral disc formed from a non-bony, fibro-cartilage material that provides for cushioning and dampening of compressive forces applied to the spinal column. The vertebral canal containing the delicate spinal cords and nerves is located just posterior to the vertebral bodies.

Various types of spinal column disorders are known and include scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in a lumbar or cervical spine) and other disorders caused by abnormalities, disease or trauma, such as ruptured or slipped discs, degenerative disc disease, fractured vertebra, and the like. Patients suffering from such conditions usually experience extreme and debilitating pain as well as diminished nerve function.

The present invention involves a technique commonly referred to as spinal fixation whereby surgical implants are used for fusing together and/or mechanically immobilizing adjacent vertebrae of the spine.

Spinal fixation may also be used to alter the alignment of the adjacent vertebrae relative to one another so as to alter the overall alignment of the spine. Such techniques have been used effectively to treat the above-described conditions and, in most cases, to relieve pain suffered by the patient. However, as will be set forth in more detail below, there are some disadvantages associated with current fixation devices.

One particular spinal fixation technique includes immobilizing the spine by using orthopedic rods, commonly referred to as spine rods, running generally parallel to the spine. This is accomplished by exposing the spine posterior and fastening bone screws to the posteriorly projecting pedicles of the appropriate vertebrae. The pedicle screws are generally placed two per vertebra, one at each pedicle on either side of the spinous process, and serve as anchor points for the spine rods. Clamping elements adapted for receiving a spine rod therethrough are then used to join the spine rods to the pedicle screws. The aligning influence of the rods forces the spine to conform to a more desirable shape. In certain instances, the spine rods may be bent to achieve a desired localized curvature of the spinal column.

This invention relates generally to improvements in spinal fixation devices of the type designed particularly for human implantation, to maintain the adjacent spinal vertebrae in a substantially fixed and predetermined spatial relation while, if desired, promoting bone ingrowth and fusion therebetween. More particularly, this invention relates to an improved pedicle screw system including a plurality of multi-angle or poly-axial pedicle screw units in combination with an elongated and interconnecting stabilizer rod which is offset relative to the pedicle screws to provide a low profile configuration designed for facilitated surgical implantation and for reduced post-operative tissue irritation.

U.S. Pat. No. 5,474,555 discloses one form of a pedicle screw device with a multi-axial receiving member or bracket. In this system, an elongated stabilizer rod is compressed by a securing member including a compression cap and a threaded nut for bearing engagement directly against an underlying pedicle screw. This system beneficially accommodates facilitated implantation wherein both the stabilizer rod and the securing member are loaded into the exposed or open top of the receiving member or bracket. However, one major drawback is that these components are stacked on top of each other, with the securing member on top of the stabilizing rod which is in turn disposed on top of the pedicle screw, all compressed in a stack within the receiving member or bracket. This results in an assembled system having a significant vertical stack-up dimension which can lead to post-surgical irritation of muscle and other patient tissue. Additionally, since the stabilizer rod must compress against the pedicle screw for proper fixation, there is no means or option for securing the stabilizer rod without also locking the pedicle screw in position relative to the receiving member.

Another concept for securing vertebrae with pedicle screws and an interconnected stabilizer rod is disclosed in U.S. Pat. 6,187,005. In this concept, the stabilizer rod is supported within a housing or bracket element at a position offset to one side of the pedicle screw, thereby beneficially reducing the overall height of the implanted system. While this results in reduced post-operative patient tissue irritation, there are disadvantages. By way of example, in most embodiments, the elongated stabilizing member is loaded into the housing element from the side, thereby making it difficult to place intra-operatively and also requiring a surgical incision or increased size to accommodate such side-loading. In addition, the disclosed system requires two separate two securing members for respectively and independently fixating the associate pedicle screw and the stabilizer rod. This use of multiple securing members undesirably increases the surgical complexity and thereby inherently increases the time needed to perform the surgical procedure.

The present invention provides a means of securing two vertebrae with respective pedicle screws in a manner providing a low profile and substantially minimal component stack-up consistent with a substantially minimized or surgical incision. The components are all designed for top-loading, and suitable fixation or securement using a single securing member such as a set screw associated with each pedicle screw. The stabilizer rod is supported within an anchor bracket at a position offset to the side of each pedicle screw, and is also located off-axis relative to the associated securing member. Additionally, the present device or system provides secure fixation of the stabilizer rod in a manner permitting limited displacement, if desired, of the associated pedicle screw relative to the anchor bracket.

Hence, the present invention offers an advantage over prior art by reducing the overall height of the implanted system, which leads to improved patient comfort with reduced tissue irritation. Moreover, the improved fixation system allows for a simple, single securing member to fixate both the stabilizer rod and the associated pedicle screw. In addition, the present device is a top-loading device, and can therefore be implanted through a smaller incision than previous examples. Finally, the present invention allows for a dynamic bone screw that can provide additional anatomic settling of the vertebrae about the inter-body device which can lead to a stronger, faster fusion.

### SUMMARY OF THE INVENTION

In accordance with the invention, an improved spinal fixation system is provided for human implantation into a pair of adjacent vertebrae, to restore and maintain the spinal anatomy in a predetermined and substantially fixed spaced relation. In this regard, the improved fixation device of the present invention is designed for use in addressing clinical problems indicated by surgical treatment of bone fractures, skeletal non-unions, weak bony tissue, degenerative disc disease, discogenic back pain, scoliosis (abnormal lateral curvature of the spine), kyphosis (abnormal forward curvature of the spine, usually in the thoracic spine), excess lordosis, and spondylolisthesis.

The improved fixation system comprises a plurality of bone anchors such as pedicle screw units and an interconnecting elongated stabilizing member such as a stabilizer rod formed from a bio-compatible material composition. Each of the bone anchors or pedicle screw units includes a pedicle bone screw having a head captured within a receiving member or anchor bracket, where it is allowed to rotate freely during its insertion into the patient bone. A first pedicle screw, with captive anchor bracket, is placed into the protruding pedicle of a first vertebral body. A second pedicle anchor, again with associated captive anchor bracket, is additionally placed into the protruding pedicle of a second and typically adjacent vertebral body. Third and subsequent bone anchors each with associated pedicle screw and captive anchor bracket may be used as desired or required.

Each bone screw is captured within the respective anchor bracket with a threaded screw shank fastened securely into patient bone, and a head of the bone screw being positioned within the bracket. The head of the bone screw is generally spherical, as is an internal mating part-spherical surface of the anchor bracket surrounding a lower screw port or bore formed therein and through which the screw shank extends for connection to patient bone. This geometry allows for axial rotation of the bone screw during threaded installation into the vertebral bone, as well as multi-angle or poly-axial articulation and orientation of the bone screw relative to the anchor bracket. The bracket has a generally open top side to allow a suitable instrument or tool to engage and drive said bone screw into the vertebral body.

Each anchor bracket further defines a generally U-shaped and upwardly open channel or trough for accepting the elongated stabilization member such as a stabilizer rod. This U-shaped trough is defined by a wing or arm protruding laterally from the lower bore and the spherical screw head seated therein, thereby forming the trough in laterally offset relation to the associated and adjacent bone screw. The stabilizer rod is placed into and seated within the troughs of the anchor brackets associated with multiple pedicle screw units. Such rod placement is aided by the fact that the anchor brackets are able to undergo limited articulation about the heads of the respective bone screws. Additionally, the placement of the stabilizer rod is aided by the orientation of the U-shaped troughs, namely, by orienting the troughs to open in an upward or dorsal direction for quick and easy drop-in placement by the surgeon.

Subsequently, both the stabilization member and the bone anchors are secured in place relative to the associated brackets by a single securement member such as a locking or set screw fastened into each bracket. In the preferred embodiment, a set screw is threaded into an internally threaded upper bore formed in the top of the associated anchor bracket at a position overlapping or at least partially overlying both the stabilizer rod and the head of the associated bone screw. With this geometry, the set screw can be advanced within the upper bore to bear with parallel applied forces against both the bone screw and the stabilizer rod, substantially without pressing the stabilizer rod against the bone screw. As a result, the set screw bears against and retains the bone screw in place within the associated anchor bracket, thereby restricting or preventing relative motion between these components. In addition, the set screw independently bears against and retains the stabilizer rod in place within the same anchor bracket, thereby preventing or preventing relative motion between these components.

In alternative embodiments, the anchor bracket may include a movable tab interposed between the set screw and the stabilizer rod, wherein advancement of the set screw within the upper bore displaces the tab against the stabilizer rod to retain said rod in place within the bracket trough. The tab may be formed integrally with the anchor bracket, or provided as a separate component mounted thereon.

The thus-formed fixation device creates a low-profile implant due to the fact that the stabilization member is located at a laterally offset position at one side, and not on top of the associated bone screw. Additionally, since a single locking screw is used to secure these components in place relative to the anchor bracket, the fixation system and method are fast and easy to use. Finally, because all components, the bone screw with accompanying anchor bracket, stabilization member, and locking screw, are placed from the same approach, namely, generally a dorsal approach, only one incision is required, and that incision may be made smaller than otherwise required for a device having a laterally positioned stabilization member.

In accordance with a further aspect of the invention, the spinal fixation system of the present invention exhibits a secondary benefit due to the fact that the bone screw and stabilization member are independently locked by the common locking or set screw. More particularly, by utilizing the locking screw to retain the stabilizer rod without pressed engagement between the stabilizer rod and the associated bone screw, a semi-constrained system is created wherein, in one embodiment, the set screw can remain at least slightly disengaged from the associated bone screw to permit relative articulation between the bone screw and anchor bracket. Such articulation, limited by the constrained stabilizer rod, can provide appropriate spacing between the thus-constrained vertebral bodies while allowing for continuous load sharing of any bone grafting material used for fusion ingrowth relative to said vertebral bodies.

Other features and advantages of the invention will become more apparent from the following detailed description, taken in conjunction with the accompanying drawings which illustrate, by way of example, the principles of the invention.

### BRIEF DESCRIPTION OF THE DRAWINGS

The accompanying drawings illustrate the invention. In such drawings:

FIG. 1 is a fragmented perspective view depicting the spinal fixation system of the present invention implanted into adjacent vertebral bodies of the human spine;

FIG. 2 is an enlarged and partially fragmented perspective view of the spinal fixation system;

FIG. 3 is an exploded perspective view of the components shown in FIG. 2;

FIG. 4 is an enlarged and fragmented side view elevation view of the spinal fixation system, taken generally on the line 4-4 of FIG. 2;

FIG. 5 is an enlarged and fragmented vertical sectional view taken generally on the line 5-5 of FIG. 2;

FIG. 6 is an enlarged and fragmented vertical sectional view similar to FIG. 5, but depicting one alternative preferred form of the invention using a stabilizer rod having a generally I-beam cross sectional shape;

FIG. 7 is another enlarged and fragmented vertical sectional view similar to FIG. 5, but showing another alternative preferred form of the invention using a stabilizer bar of grooved configuration;

FIG. 8 is an enlarged and fragmented sectional view taken generally on the line 8-8 of FIG. 7;

FIG. 9 is an enlarged and fragmented sectional view similar to FIG. 8, but depicting an alternative version thereof;

FIG. 10 is an enlarged and fragmented perspective view generally similar to FIG. 1, but showing yet another alternative preferred embodiment of the invention;

FIG. 11 is an enlarged and fragmented perspective view of the device shown in FIG. 10, with the stabilizer rod and locking member removed to illustrate internal construction details;

FIG. 12 is an enlarged and fragmented sectional view taken generally on the line 12-12 of FIG. 10;

FIG. 13 is an enlarged and fragmented sectional view similar to FIG. 12, but depicting an alternative version thereof;

FIG. 14 is a fragmented perspective view of a further another preferred form of the invention; and

FIG. 15 is an enlarged and fragmented vertical sectional view taken generally on the line 15-15 of FIG. 14.

### DETAILED DESCRIPTION OF THE PREFERRED EMBODIMENTS

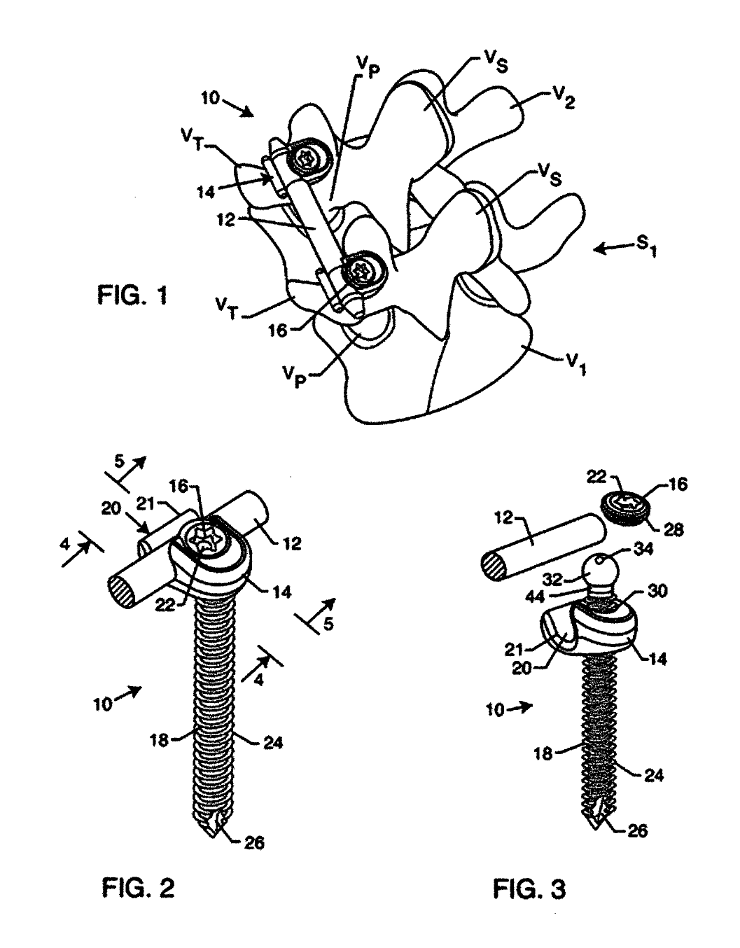

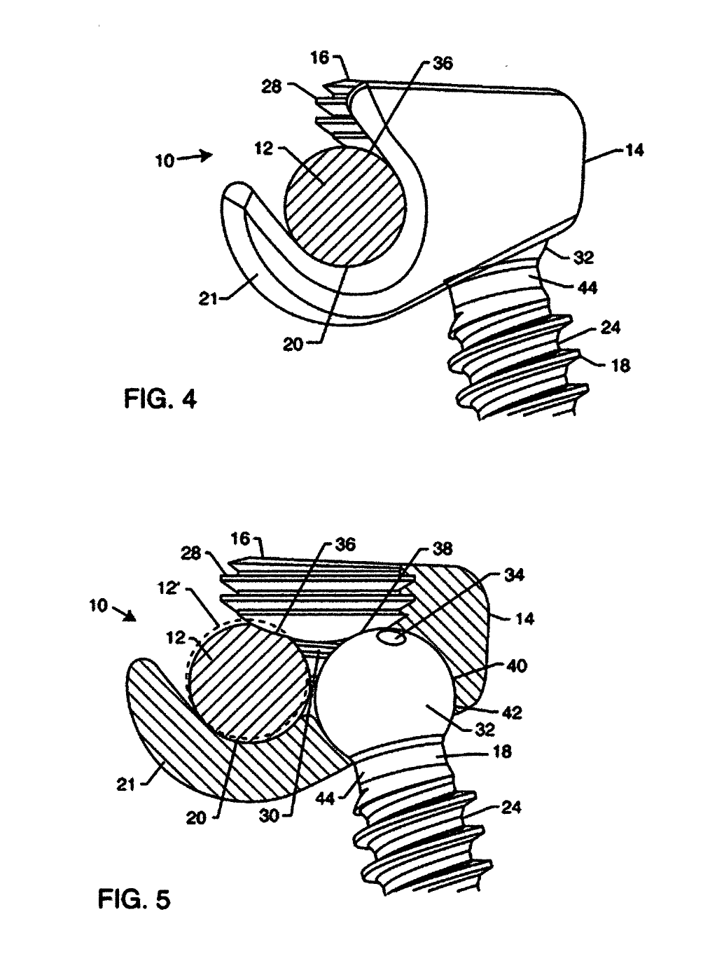

As shown in the exemplary drawings, an improved spinal fixation device referred to generally in FIGS. 1-5 by the reference numeral 10 is provided for attachment to at least a pair of adjacent patient bones such as spinal vertebrae S₁ (FIG. 1) to maintain the skeletal structures in spaced relation while promoting bone ingrowth and fusion. In general, the improved fixation device 10 comprises a bio-compatible stabilization member such as an elongated stabilizer rod 12 providing a strong mechanical load bearing structure, with said rod 12 connecting with a plurality of bone screw units each including a bone screw 18 secured or anchored to the vertebrae S₁.

Each bone screw 18 comprises a threaded shank portion 24 for engaging and securely anchoring to patient bone. In the preferred embodiment, each bone screw 18 is manufactured from a high strength bio-compatible material, allowing for load carrying capabilities. Proximal to the threaded portion 24 is a screw head or head section 32 that is designed to seat inside a lower internal bore 40 of a receiving member or anchor bracket 14. The head 32 of the screw 18 is generally spherical in nature for substantially mated articulatory fit with a part-spherical seat defining the lower bore 40 formed in the anchor bracket 14, thereby accommodating limited angular articulation of the bone screw 18 within and relative to the bracket 14. As shown, the elongated threaded shank 24 of the bone screw 18 is fitted downwardly through the lower bore 40 formed in the bracket 14, to seat the screw head 32 within and against the part-spherical shaped bore 40. In this position, an upper margin of the screw head 32 lies below an upper margin of the bracket 14.

The anchor bracket 14 also defines a trough or channel 20 for receiving, supporting, and retaining the stabilizer rod 12. This trough 20 is defined by a laterally projecting arm or wing 21 disposed laterally or in offset relation to one side of the part-spherical bore 40 and the bone screw head 32 seated therein. Additionally, the bracket 14 includes or defines an upper internally threaded bore 30 positioned for at least partially overlying both the head 32 of the bone screw 18 seated within the lower bore 40 as well as the trough 20 for receiving the stabilizer rod 12. As shown best in FIG. 5, and in accordance with a preferred form of the invention, a vertical centerline through this upper threaded bore 30 extends generally between the lower bore 40 and the trough 20, whereby the peripheral margin of the upper bore 30 at least partially overlies and intersects both the bone screw-receiving lower bore 40 (and the bone screw head 32 seated therein) and the rod-receiving trough 20.

The threaded upper bore 30 formed in the anchor bracket 14 receives a locking or securement member such as a set screw 16. Said locking or set screw 16, when threaded into the upper bore 30, has a lower face seated against and compressively engaging both the stabilizer rod 12 and the bone screw head 32. Accordingly, the set screw 16 creates a friction or compression lock between the bracket 14 and the rod 12, while at the same time creating a friction or compression lock between the bracket 14 and the head 32 of the bone screw 18.

FIG. 1 shows the preferred fixation device 10 attached to a skeletal structure S1, specifically the vertebrae of the lumbar spine. Each of the bone screw units comprises a pedicle bone screw 18 anchored into a respective one of the protruding pedicles V_{P} of the spine S₁. In order to stabilize the spine S₁, the pedicle bone screws 18 are connected together via the stabilizing rod component 12 and the respective anchor brackets 14. The stabilizer rod 12 runs adjacent to the axis of the spine, lateral of or offset to the spinous processes V_{S}, and medial of the transverse processes V_{T}. It is in this area that autologous bone or other suitable graft or fusion material is typically placed in an attempt to fuse the adjacent vertebrae together. The stabilizer rod 12 is effectively locked within the multiple anchor brackets 14 by means of the respective locking set screws 16. However, the stabilizer rod 12 is not compressed against the associated bone screws 18.

The improved fixation device or system 10 shown in FIGS. 2-5 comprises four major components, namely, the stabilizer rod 12, and at least two pedicle screw units each including the pedicle screw 18, the associated anchor bracket 14, and the locking set screw 16. The rod 12 generally comprises an elongated cylindrical body connected to two or more of the pedicle screw units. The stabilizer rod 12 is received and locked into the bracket 14 of each pedicle screw unit within the upwardly or dorsally open trough or channel 20 of generally U-shaped configuration.

As previously noted, each pedicle bone screw 18 has a threaded shank portion 24 for secure attachment to adjacent bone structure. Additionally, these threads may have a self-tapping feature 26 to allow quicker insertion into the bone. The head 32 of the bone screw 18, being generally spherical in shape, is captured within the part-spherical lower bore 40 within the anchor bracket 14. Maintaining the screw 18 in the bracket 14 is a smaller bore 42 (FIG. 5) at the distal end or underside of the part-spherical bore 40. The smaller bore 42 is larger than a neck 44 of the screw 18, but is smaller than the head 32. The bone screw 18 is allowed to articulate within and about the part-spherical lower bore 40 in relation to the bracket 14. On the proximal end of the screw head 32 is a drive feature 34 such as a hex recess for receiving a drive tip of a suitable installation tool (not shown) to facilitate insertion of the screw 18 into the bone.

The internally threaded upper bore 30 is formed on the anchor bracket 14 at a location spaced above the lower, part-spherical bore 40 and the laterally offset wing or arm 21 forming the trough 20, thereby defining an internal chamber for receiving and supporting the screw head 32 in laterally offset relation with the stabilizer rod 12 within the trough, as will be described in more detail. This upper bore 30, in the preferred geometry as shown, at least partially overlies both the lower bore 40 and the trough 20, and may be defined by a laterally open-sided, part-circular bore having a sufficient circumferential span (greater than 180 degrees) for receiving and retaining the locking set screw 16. As shown, the open side of the upper bore 30 is presented toward and generally partially overlies the trough 20, thereby insuring that a portion of the set screw overlies the trough 20 for engaging and locking with the stabilizer rod 12 seated therein.

The locking set screw 16 is threadably inserted into the threaded bore 30. Threads 28 of the set screw 16 are designed to prevent said screw 16 from backing out of the threaded bore 30. An upper side of the set screw 16 has a drive feature 22 such as a hex-shaped recess formed therein for receiving a tool tip of a suitable installation tool (also not shown) to allow sufficient torque to be applied to said screw 16. As the set screw 16 is threadably advanced into the threaded bore 30 of the anchor bracket 14, a lower or underside face of the set screw 16 engages and presses against the stabilizer rod 12 seated within the associated trough 20. In doing so, the set screw 16 forces the rod 12 against the trough 20 in a generally diametric direction corresponding with a contact point 36 between the set screw 16 and the rod 12. Furthermore, the set screw 16 engages and presses against the spherical head 32 of the bone screw 18 at a contact point contact point 38, thereby forcing said head 32 against the part-spherical seat defining the bore 40 of the anchor bracket 14. These actions effectively lock both the stabilizer rod 12 and the bone screw 18 in place relative to the anchor bracket 14.

By coupling the stabilizer rod 12 between multiple pedicle screw units which are in turn secured respectively to different, typically adjacent vertebrae (as viewed in FIG. 1), the stabilizer rod 12 effectively limits or precludes intervertebral motion. Autologous bone or other suitable graft or fusion material can be placed into the space between these adjacent vertebrae in an attempt to fuse them together.

In accordance with one important aspect of the invention, the assembled system comprises the pedicle screw units and related stabilizer rod presents a minimally low implantation profile for reducing or eliminating post-operative patient discomfort attributable to tissue irritation. In addition, the system is adapted for surgical implantation via a surgical incision of minimal size, since all of the components are installed and accessed from a common direction. That is, each pedicle bone screw 18 and associated anchor bracket 14 is installed by dorsal access from above, followed by similar installation of the stabilizer rod 12 quickly and easily into the dorsally or upwardly open trough 20 of each pedicle screw unit. Then, the locking set screws 16 are similarly installed by dorsal access from above, with a single set screw 16 performing the dual functions of engaging and locking both the bone screw 18 and the stabilizer rod 12. Importantly, and consistent with maintaining the desired minimal system profile, the bone screw head 32 and the stabilizer rod 12 are not compressively engaged with each other.

In one alternative preferred embodiment of the device 10 shown and described with respect to FIGS. 1-5, the locking set screw 16 may engage and lockingly retain only the stabilizer rod 12 but not the head 32 of the underlying bone screw 18. In this regard, such alternative configuration can be obtained by using a stabilizer rod 12′ (shown in dashed lines in FIG. 5) of comparatively larger diameter, thereby moving contact point thereof with the lower face of the set screw 16 in a manner assuring at least a minor spacing between the set screw 16 and the bone screw head 32. In the absence of compressive contact between the set screw 16 and the bone screw head 32, the bone screw head 32 is able to articulate within the part-spherical bore 40 relative to the anchor bracket 14. This arrangement allows for dynamic fixation, since the stabilizer rod 12 is locked, but the pedicle screw 18 is not. The rod 12 thereby maintains spacing between adjacent anchor brackets 14 of adjacent pedicle screw units, but the screws 18 may undergo a minor degree of restricted motion relative to the associated anchor brackets 14. Such minor articulation can be an important factor in obtaining optimal fusion attachment of the adjacent vertebrae. The motion is restricted by the relationship between the small bore 42 in the anchor bracket 14, and the neck 44 of the screw 18.

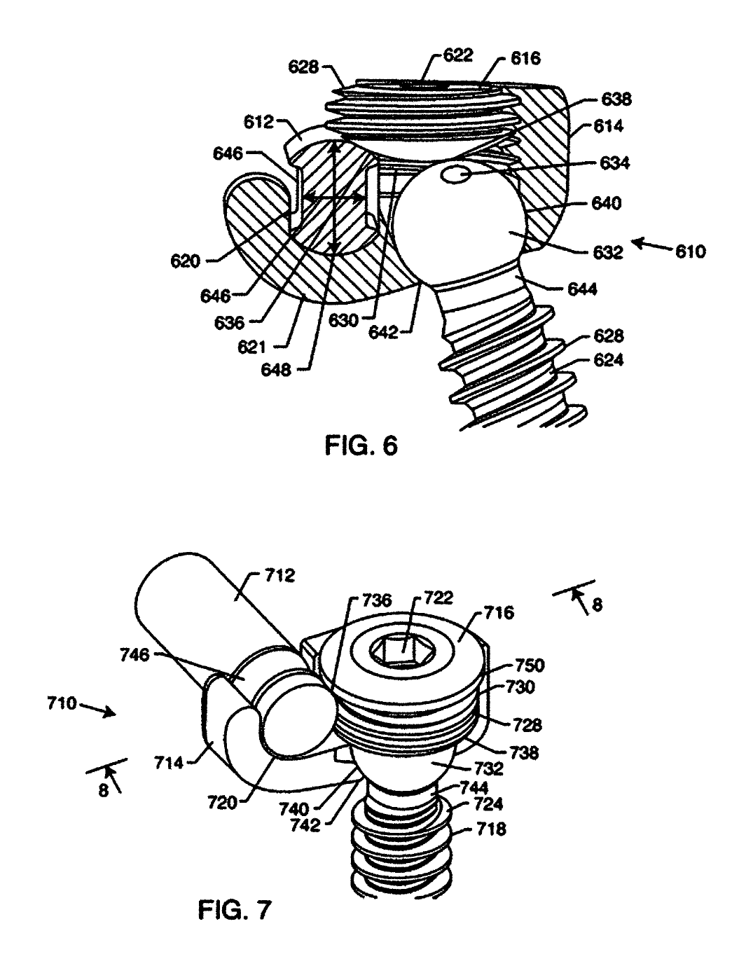

FIG. 6 shows a further alternative embodiment of the improved spinal fixation device or system, wherein components corresponding with those previously shown and described in FIGS. 1-5 are identified by common reference numerals increased by 600. As shown, the modified fixation device 610 generally conforms with the embodiment shown in FIGS. 1-5, except that a modified stabilizer rod 612 is provided with a non-circular cross sectional shape in the configuration of an I-beam. The rod 612 is oriented within the associated trough 620 of each pedicle screw unit with a long axis 648 of the rod 612 oriented generally vertically or anterior-posterior to insure that the thinnest portion 646 thereof is presented or captured in a medial-lateral X-ray image.

As previously shown and described, the stabilizer rod 612 can be locked in place within the trough 620 by means of the locking set screw 616, wherein this set screw 616 also engages and locks the head 632 of an associated bone screw 618 in place relative to the associated anchor bracket 614. Alternatively, if desired, the set screw 616 may engage and lock only the rod 612, while remaining in at least slightly spaced relation to the screw head 632 to accommodate limited articulation thereof relative to the anchor bracket 614.

More specifically, each bone screw 618 has a threaded potion 624 which secures it to the bone. The head 632 of the bone screw 618, being generally spherical, is captured within a part-spherical lower bore 640 within the anchor bracket 614. Maintaining the screw 618 in the bracket 614 is a smaller bore 642 at the distal end of the lower bore 640. The smaller bore 642 is larger than the neck 644 of the screw 618, but is smaller than the head 632. The bone screw 618 is allowed to articulate about this lower bore 640 in relation to the anchor bracket 614. On the proximal end of the screw head 632 is a drive feature 634 such as a hex recess to facilitate insertion of the screw 618 into the bone.

An internally threaded and open-sided upper bore 630 on the anchor bracket 614 intersects both the trough 620 and the part-spherical lower bore 640. The locking set screw 616 is inserted into the threaded bore 630. The threads 628 of the locking screw 616 are designed to prevent said screw from backing out. The locking screw 616 has a drive feature 622 such as a hex recess to allow sufficient torque to be applied to said screw. As the locking screw 616 advances into the threaded bore 630 of the anchor bracket 614, it presses against the rod member 612. In doing so, it forces the rod 612 against the trough 620 and the contact point 636 of the screw 616 and rod 612. The trough 620 is shaped to receive, support and maintain the orientation of the rod 612 to ensure that the tallest portion 648 of the rod 612 is in contact 636 with the lock screw 616. Furthermore, the locking screw 616 is shown making contact 638 with the bone screw head 632, forcing said head against the part-spherical bore 640 of the anchor bracket 614. These actions lock both the rod 612 and the bone screw 618 in place.

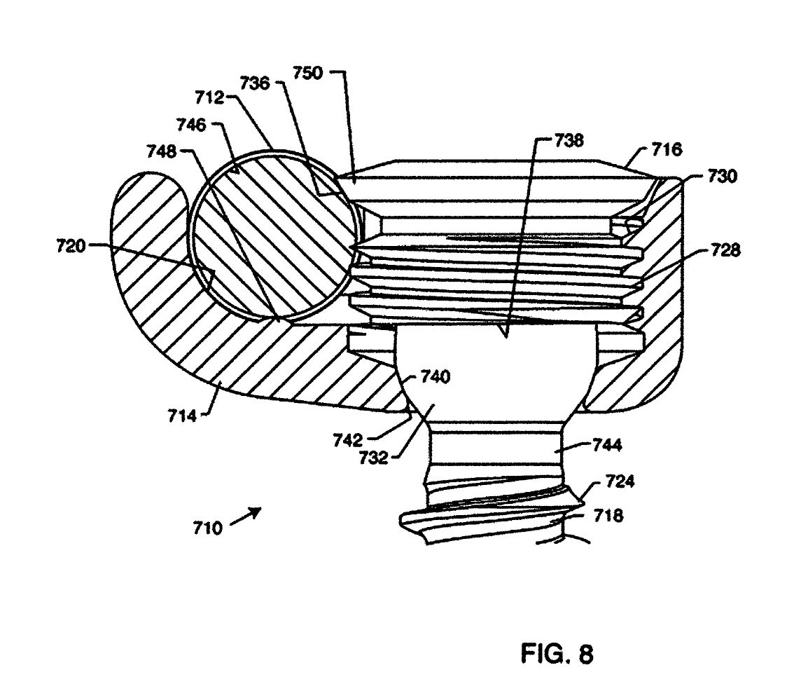

A further alternative preferred form of the invention is shown in FIGS. 7-8, wherein components corresponding to those previously shown and described in FIGS. 1-5 are identified by common reference numbers increased by 700. As shown, the modified system or device 710 again comprises four major components, namely, a rod component 712, and a plurality of pedicle screw units each including a bone screw member 718, an anchor bracket 714, and a locking screw 716. The rod component 712 is a generally elongated cylindrical body which connects two or more bone screws 718. The rod 712 is received by the anchor bracket 714 of each pedicle screw unit with an upwardly open, generally U-shaped trough 720. The rod 712 has circumferential grooves 746 (one of which is shown in FIGS. 7-8) formed therein in the cylinder surface for mating with a protrusion or bump 748 (FIG. 8) formedin the receiving trough 720. Furthermore, the grooves 746 engage an upper non-threaded flange portion 750 of the locking set screw 716.

Each bone screw 718 has a threaded portion 724 which secures it to the bone. The head 732 of the bone screw 718, being generally part-spherical, is captured within a part-spherical lower bore 740 formed within the anchor bracket 714. Maintaining the screw 718 in the anchor bracket 714 is a smaller bore 742 at a distal end of the part-spherical bore 740. The smaller bore 742 is larger than the neck 744 of the screw 718, but is smaller than the head 732. The bone screw 718 is allowed to articulate about this lower bore 740 in relation to the anchor bracket 714.

An internally threaded and open-sided upper bore 730 on the anchor bracket 714 intersects both the trough 720 and the part-spherical bore 740, in the same manner as previously shown and described herein. A locking screw 716 is inserted into the threaded bore 730. The threads 728 of the locking screw 716 are designed to prevent said screw from backing out. The locking screw 716 has a drive feature 722 such as a hex recess to allow sufficient torque to be applied to said screw. As the locking screw 716 advances into the threaded upper bore 730 of the anchor bracket 714, it presses against the rod component 712. The non-threaded portion 750 of the screw 716 engages the associated groove 746 on the rod 712. In doing so, it forces the rod 712 against the trough 720 and the contact point 736 (FIG. 7) of the screw 716 and rod 712. The groove 746 of the rod 712 receives and engages the protrusion or bump 748 in the trough 720 (FIG. 8).This aids in restricting axial movement of the rod 712 which may dislodge the locking screw 716. Furthermore, the locking screw 716 makes contact 738 with the bone screw head 732, forcing said head against the spherical bore 740 of the anchor bracket 714. These actions lock both the rod 712 and the bone screw 718 in place.

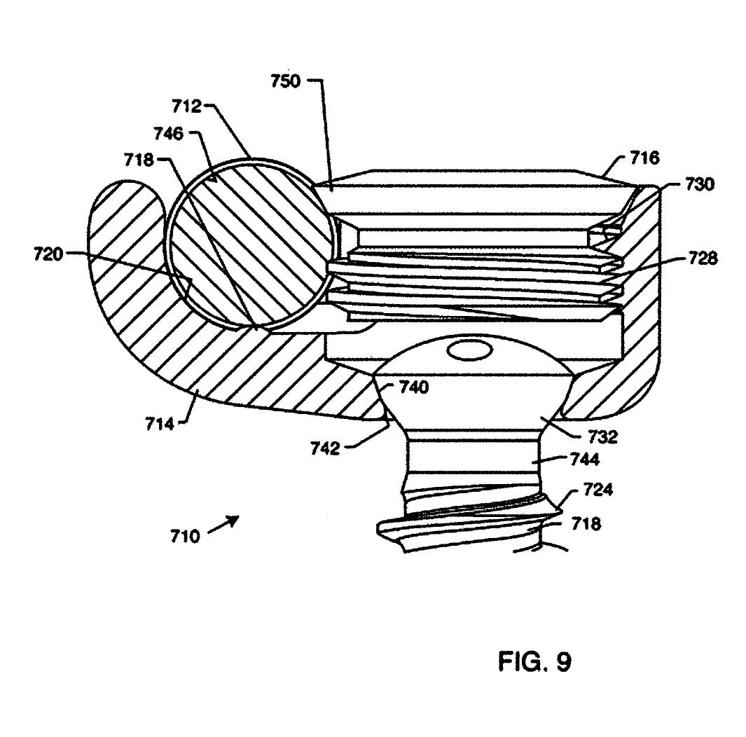

In an alternative preferred embodiment of the device 710 in FIGS. 7-8, the non-threaded portion 750 of the lock screw 716 makes contact 736 only with the grooves 746 of the rod 712, but the lock screw 716 does not make compressive contact with the bone screw head 732. Since there is no contact at point 738, the bone screw head 732 is still able to articulate about the part-spherical bore 740 of the anchor bracket 714. This allows for dynamic fixation, since the rod 712 is locked, but the screw 718 is not. The rod 712 thereby maintains spacing between adjacent anchor brackets 714, but the screws 718 still have some restricted motion. The motion is restricted by the relationship between the small bore 742 in the anchor bracket 714, and the neck 744 of the screw 718. This alternative configuration is shown in FIG. 9.

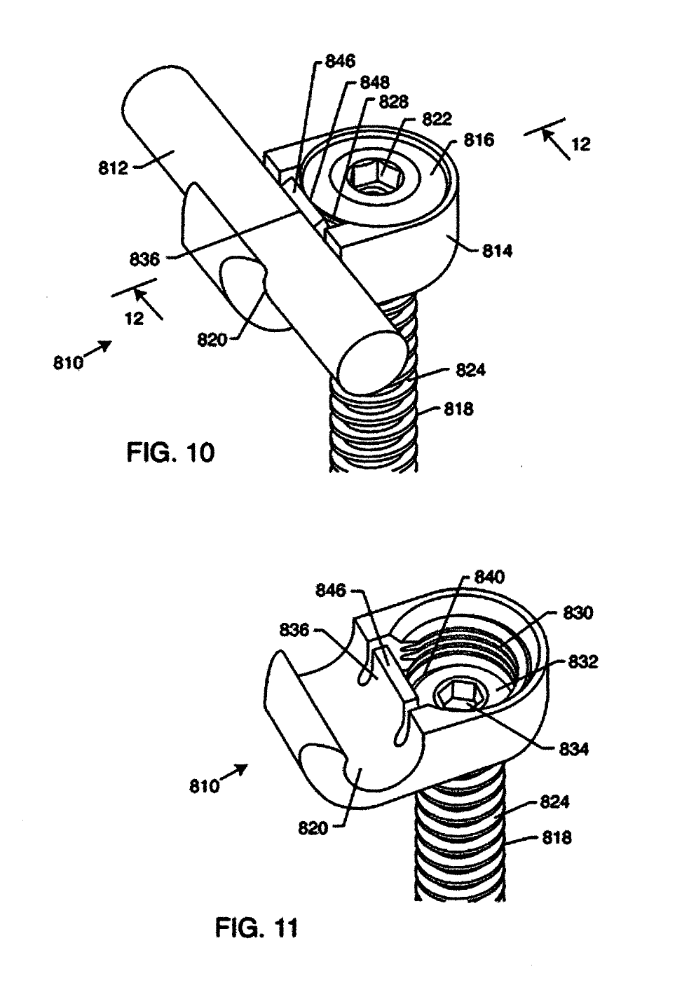

Another alternative preferred form of the invention is shown in FIGS. 10-12, wherein components corresponding to those previously shown and described in FIGS. 1-5 are identified by common reference numbers increased by 800. As shown a modified spinal fixation device or system 810 is shown and again comprises four major components, namely, a rod component 812, and pedicle screw units each including a bone screw member 818, an anchor bracket 814, and a locking set screw 816. The rod component 812 is generally an elongated cylindrical body connected between two or more of the pedicle screw units which are secured to patient bone by their respective bone screws 818. The rod 812 is received by the anchor bracket 814 into a generally U-shaped trough 820. Adjacent to the trough 820 at an inboard side disposed between the trough 820 and an internally threaded bore 830 is a flexible or movable tab 846 formed integrally with the anchor bracket 814.

The bone screw 818 has a threaded potion 824 which secures it to the bone. The head 832 of the bone screw 818, being generally spherical, is captured within a part-spherical lower bore 840 formed within the anchor bracket 814. Maintaining the screw 818 in the anchor bracket 814 is a smaller bore 842 at a distal end of the part-spherical bore 840. The smaller bore 842 is larger than the neck 844 of the screw 818, but is smaller than the head 832. The bone screw 818 is allowed to articulate about this lower bore 840 in relation to the anchor bracket 814.

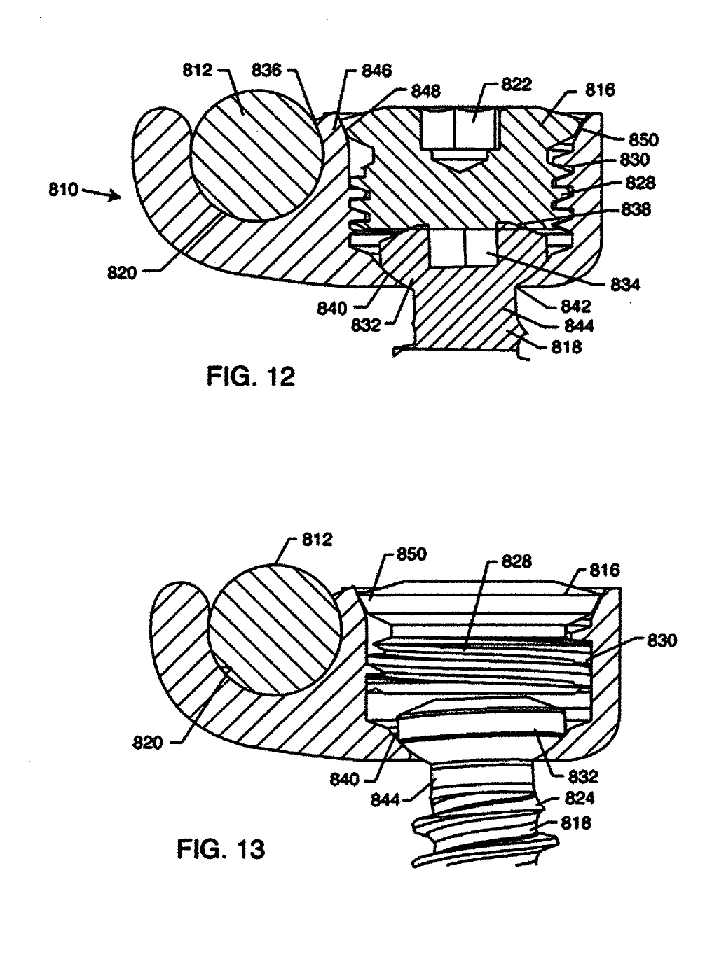

An internally threaded upper bore 830 on the anchor bracket 814 intersects with the spherical bore 840. The thread within this upper bore 830 has a part-circumferential configuration similar to the embodiments shown and described in FIGS. 1-7, but instead of an open-sided geometry the tab 846 upstands between the bore 830 and the trough 820. A locking set screw 816 is inserted into the threaded upper bore 830. The threads 828 of the locking screw 816 are designed to prevent said screw from backing out. The locking set screw 816 has a drive feature 822 such as a hex recess to allow sufficient torque to be applied to said screw. As the locking screw 816 advances into the threaded bore 830 of the receiving member 814, it forces the tab 846 to displace laterally outwardly (as indicated by arrow 848) against the rod component 812 with an upper margin of the tab 846 engaging the rod 812 at a location above a horizontal centerline thereof. A non-threaded upper flange portion 850 (FIG. 12) of the screw 816 engages the tab 846 so as not to damage the threads 828. In doing so, the tab 846 forces the rod 812 against the trough 820 and the contact point 836 of the tab 846 and rod 812. This tab 846 aids in restricting axial rotation of the locking screw 816 which may dislodge said locking screw. Furthermore, the locking screw 816 makes contact 838 with the bone screw head 832, forcing said head against the spherical bore 840 of the anchor bracket 814. These actions lock both the rod 812 and the bone screw 818 in place.

Additionally, with the device 810 in FIGS. 10-12, the non-threaded portion 850 of the lock screw 816 may make contact 848 only with the tab 846 in turn pressing against the of the rod 812 and but not with the bone screw head 832. The variation is depicted in FIG. 13. Since there is no contact at point 838, the bone screw head 832 is still able to articulate about the part-spherical bore 840 of the anchor bracket 814. This allows for dynamic fixation, since the rod 812 is locked, but the screw 818 is not. The rod 812 thereby maintains spacing between adjacent anchor brackets 814, but the screws 818 still have some restricted motion. The motion is restricted by the relationship between the small bore 842 in the anchor bracket 814, and the neck 844 of the screw 818.

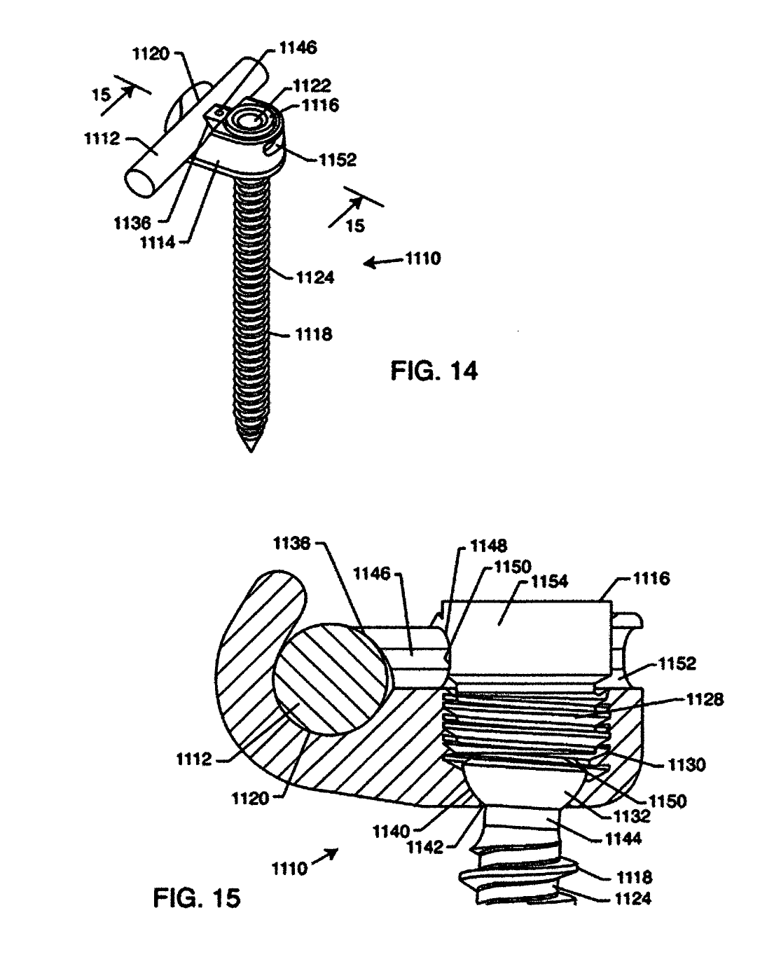

A still further alternative preferred form of the invention is shown in FIGS. 14-15, wherein components corresponding to those previously shown and described in FIGS. 1-5 are identified by common reference numbers increased by 1100. As shown, the improved spinal fixation device or system 1110 comprises the same group of four major components, namely, a rod component 1112, and a pedicle screw unit including a bone screw member 1118, an anchor bracket 1114, and a locking set screw 1116. In this embodiment, however, an additional component in the form of a separate lock tab 1146 is provided at an inboard side of the trough 1120 between the trough and an internally threaded bore 1130. The rod member 1110 is generally an elongated cylindrical body which is connected between at least two pedicle screw units which are in turn secured to patient bone by means of their respective bone screws 1118. The rod 1112 is received by the anchor bracket 1114 into the trough 1120 of generally U-shaped configuration. Adjacent to the trough 1120 is the lock tab 1146 that is located between the threaded bore 1130 and said trough.

The bone screw 1118 has a threaded potion 1124 which secures it to the bone. The head 1132 of the bone screw 1118, being generally spherical, is captured within a part-spherical lower bore 1140 within the anchor bracket 1114. Maintaining the screw 1118 in the anchor bracket 1114 is a smaller bore 1142 at a distal end of the part-spherical bore 1140. The smaller bore 1142 is larger than the neck 1144 of the screw 1118, but is smaller than the head 1132. The bone screw 1118 is allowed to articulate about this lower bore 1140 in relation to the anchor bracket 1114.

An internally threaded upper bore 1130 on the anchor bracket 1114 intersects with the spherical bore 1140 and is adjacent to the lock tab 1146. A locking set screw 1116 is inserted into the threaded bore 1130. The threads 1128 of the locking screw 1116 are designed to prevent said screw from backing out. The locking screw 1116 has a drive feature 1122 such as a hex recess to allow sufficient torque to be applied to said screw. As the locking screw 1116 advances into the threaded bore 1130 of the anchor bracket 1114, it forces the tab 1146 as indicated by arrow 1148 to slide laterally against the rod member 1112. A non-threaded upper shank portion 1150 of the screw 1116 engages the tab 1146 so as not to damage the threads 1128. In doing so, the tab 1146 forces the rod 1112 against the trough 1120 and the contact point 1136 of the tab 1146 and rod 1112. This tab 1146 aids in restricting axial rotation of the locking screw 1116 which may dislodge said locking screw. Furthermore, the locking screw 1116 makes contact 1138 with the bone screw head 1132, forcing said head against the part-spherical bore 1140 of the anchor bracket 1114. These actions lock both the rod 1112 and the bone screw 1118 in place.

As an additional feature of the device or system 1110 in FIGS. 11-12, the non-threaded portion 1150 of the lock screw 1116 makes contact only with the tab 1146 which in turn slides laterally and presses against the rod 1112 but not necessarily with the bone screw head 1132. That is, the set screw 1116 can be advanced sufficiently to engage and lock the bone screw head 1132 relative to the anchor bracket 1114, or the set screw 1116 can be positioned in slight spaced relation to the bone screw head 1132 so that there is no contact at point 1138 and the bone screw head 1132 still able to articulate within the part-spherical lower bore 1140 relative to the anchor bracket 1114. This allows for static or dynamic fixation, as may be indicated by a particular patient condition.

The invention, in the various embodiments shown and described herein, thus provides a substantial improvement in addressing clinical problems indicated for surgical treatment of scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in a lumbar or cervical spine) and other disorders caused by abnormalities, disease or trauma, such as ruptured or slipped discs, degenerative disc disease, fractured vertebra, and the like.

The fixation device of the present invention provides at least the following benefits over the prior art:
lower profile;
more secure fixation; and
the option of dynamic fixation

A variety of further modifications and improvements in and to the fixation device of the present invention will be apparent to those persons skilled in the art. Accordingly, no limitation on the invention is intended by way of the foregoing description and accompanying drawings, except as set forth in the appended claims.

What is claimed is:

1. A spinal fixation system for the stabilization of one or more spinal bone segments, comprising:
at least two bone anchors each adapted for respective attachment to patient bone, each of said bone anchors including a bone screw having a threaded shank and a head, and an anchor bracket defining a lower bore for pass-through reception of said bone screw shank, said bone screw head being larger than said lower bore for seating within said anchor bracket, said anchor bracket further defining an upwardly open trough disposed in laterally offset relation with said lower bore;
an elongated stabilizer rod for reception within said trough of each of said bone anchors; and
each of said bone anchors further including a securement member carried by said anchor bracket in a position at least partially overlying said bone screw head and for applying a compressive force to said stabilizer rod to retain said stabilizer rod within said trough and in substantially spaced relation to said bone screw head.

2. The spinal fixation system of claim 1 wherein said bone screw head has a generally part-spherical surface, and further wherein said lower bore of said anchor bracket defines a part-spherical seat for receiving and supporting said bone screw head.

3. The spinal fixation system of claim 2 wherein said securement member further applies a compressive force to said bone screw head for locking said bone screw in position relative to said anchor bracket.

4. The spinal fixation system of claim 2 wherein said securement member is in spaced relation to said bone screw head to permit articulation of said bone screw head within said anchor bracket.

5. The spinal fixation system of claim 1 wherein said securement member further applies a compressive force to said bone screw head for locking said bone screw in position relative to said anchor bracket.

6. The spinal fixation system of claim 1 wherein said securement member comprises a set screw threadably received into an internally threaded upper bore formed in said anchor bracket.

7. The spinal fixation system of claim 6 wherein said internally threaded upper bore is laterally open-sided in a direction toward said trough, said set screw at least partially overlying said trough for engaging said stabilizer rod when said set screw is received into said upper bore.

8. The spinal fixation system of claim 6 further including a tab interposed between said internally threaded upper bore and said trough, said set screw urging said tab into compressive engagement with said stabilizer rod upon threaded reception of said set screw into said upper bore.

9. The spinal fixation system of claim 6 wherein said tab is formed integrally with said anchor bracket.

10. The spinal fixation system of claim 6 wherein said tab comprises a component separate from said anchor bracket.

11. The spinal fixation system of claim 1 further including means for preventing axial displacement of said stabilizer rod within said trough of each of said bone anchors.

12. The spinal fixation system of claim 11 wherein said means for preventing axial displacement of said stabilizer rod comprises at least one protrusion formed within said trough for reception into at least one circumferential groove formed in said rod.

13. The spinal fixation system of claim 1 wherein said stabilizer rod has a generally circular cross sectional shape.

14. The spinal fixation system of claim 1 wherein said stabilizer rod has a non-circular cross sectional shape elongated in a vertical direction when said rod is seated within said trough.

15. A pedicle screw unit for attachment to a spinal bone segment and for receiving and supporting an elongated stabilizer rod, said pedicle screw unit comprising:
an anchor bracket defining a lower bore, and an upwardly open trough disposed in laterally offset relation to said lower bore;
a pedicle screw having an elongated threaded shank receivable downwardly through said lower bore, and a head having a size larger than said lower bore whereby said head is retained within said anchor bracket;
said pedicle screw shank being adapted for connection to a spinal bone segment of a patient, and said upwardly open trough being adapted to receive and support an elongated stabilizer rod therein in a position offset laterally from said pedicle screw head; and
a securement member carried by said anchor bracket in a position at least partially overlying said bone screw head and for applying a compressive force to the stabilizer rod to retain the stabilizer rod within said trough and in substantially spaced relation to said bone screw head.

16. The pedicle screw unit of claim 15 wherein said screw head has a generally part-spherical surface, and further wherein said lower bore of said anchor bracket defines a part-spherical seat for receiving and supporting said bone screw head.

17. The pedicle screw unit of claim 16 wherein said securement member further applies a compressive force to said screw head for locking said pedicle screw in position relative to said anchor bracket.

18. The pedicle screw unit of claim 16 wherein said securement member is in spaced relation to said screw head to permit articulation of said pedicle screw head within said anchor bracket.

19. The pedicle screw unit of claim 15 wherein said securement member comprises a set screw threadably received into an internally threaded upper bore formed in said anchor bracket.

20. The pedicle screw unit of claim 19 wherein said internally threaded upper bore is laterally open-sided in a direction toward said trough, said set screw at least partially overlying said trough for engaging the stabilizer rod when said set screw is received into said upper bore.

21. The pedicle screw unit of claim 19 further including a tab interposed between said internally threaded upper bore and said trough, said set screw urging said tab into compressive engagement with the stabilizer rod upon threaded reception of said set screw into said threaded bore.

22. The pedicle screw unit of claim 15 further including means for preventing axial displacement of said stabilizer rod within said trough of each of said anchors.
